(19) United States
(12) Patent Application Publication
(10) Pub. No.: US 2025/0195044 A1
(43) Pub. Date: Jun. 19, 2025

(54) CAPSULE FOR GASTROINTESTINAL SAMPLING

(71) Applicant: Eli Lilly and Company, Indianapolis, IN (US)

(72) Inventors: Sina NEJATI, West Lafayette, IN (US); Rahim RAHIMI, West Lafayette, IN (US); Mohit Singh VERMA, West Lafayette, IN (US); Jiangshan WANG, West Lafayette, IN (US)

(21) Appl. No.: 18/847,285

(22) PCT Filed: Mar. 7, 2023
(86) PCT No.: PCT/US23/14678

§ 371 Date: Sep. 16, 2024

Related U.S. Application Data
(60) Provisional application No. 63/320,825, filed on Mar. 17, 2022.

(51) Int. Cl. A61B 10/00

(52) U.S. Cl. CPC A61B 10/0045; A61B 2010/0061

(57) ABSTRACT

Passive devices for non-invasive gastrointestinal sampling are disclosed. In some embodiments, a capsule device includes a capsule housing with a sampling aperture and a biodegradable coating that seals the sampling aperture. The biodegradable coating may be disposed within the sampling aperture. The capsule contains an absorbent sampling hydrogel and a sealing member between the sampling hydrogel and the sampling aperture. The capsule is ingested by a patient. Once the capsule reaches a desired location in the gastrointestinal tract, the biodegradable coating dissolves to allow gastrointestinal fluid into the capsule to be absorbed by the sampling hydrogel. Absorption of the fluid causes the sampling hydrogel to expand, thereby pressing the sealing member against the sampling aperture to seal the capsule. In some embodiments, the biodegradable coating may have multiple layers that may dissolve at different locations in the gastrointestinal tract to facilitate targeting of particular locations, for example the colon.

### FIELD

Disclosed embodiments are related to capsules for gastrointestinal sampling and related methods of use.

### BACKGROUND

Development of new tools that can accurately sample microbial communities, proteins, chemicals, and other biomarkers of interest throughout the gastrointestinal tract (GI tract) may facilitate more effective and more accurate prediction and diagnosis of disease and disease progression.

Numerous studies have found that the gut microbiota plays an important role in the pathophysiology which dominates human health. Many of these studies have identified the effects of the gut microbiota on human metabolism, nutrition uptake, efficacy of orally-administered therapeutics, and functionality of immune and neural systems. For example, a number of studies have found correlations between microbiota imbalance (dysbiosis) and various diseases including diabetes, obesity, and metabolic syndrome; diseases which affect approximately 30 million people in the US. Similarly, recent insights regarding possible ways that gut bacteria may influence development and maintenance of the nervous system suggest a link between gut microbiome composition and the regulation of psychoneurological disorders including anxiety, depression, and dysbiosis in autism.

Furthermore, many diseases have been linked to production of different biomarkers and mRNA transcripts in the GI tract. For instance, fecal calprotectin (fCal) has been shown to be the most accurate in detection of inflammatory bowel disease (IBD) and mucosal damage. Although several studies have demonstrated associations between fCal levels and degree of inflammation, the level of calprotectin is highly dependent on several factors including diet, water content in fecal samples, and disease location. Patients with ileal Crohn's disease CD may have large ulcers with low levels of fCal. This may suggest that although fecal analysis enables a simple assessment of potential IBD, it does not indicate the possible disease location or status.

### SUMMARY

In one embodiment, a device for passive sampling of the gastrointestinal tract comprises a capsule housing bounding a cavity, a sampling aperture formed in the capsule housing and providing fluid communication between the cavity and an exterior of the capsule housing, a sampling hydrogel positioned inside the cavity. Upon exposure to a sample fluid, the sampling hydrogel is configured to absorb the sample fluid, expand within the cavity, and store the sample fluid for subsequent analysis. The device further comprises a sealing member positioned within the cavity between the sampling hydrogel and the sampling aperture. Expansion of the sampling hydrogel within the cavity presses the sealing member into engagement with the sampling aperture to seal the cavity. The device further comprises a biodegradable coating covering the sampling aperture. The biodegradable coating comprises a plurality of biodegradable coating layers. Degradation of the plurality of biodegradable coating layers exposes the sampling aperture to permit fluid flow into the cavity.

In another embodiment, a method of manufacturing a device for passive sampling of the gastrointestinal tract comprises providing a capsule housing having a cavity and a sampling aperture. The sampling aperture provides fluid communication between the cavity and an exterior of the capsule housing. The method further comprises closing the sampling aperture with a biodegradable coating comprising a plurality of biodegradable coating layers. Degradation of the plurality of biodegradable coating layers exposes the sampling aperture to permit fluid flow into the cavity.

In another embodiment, a device for passive sampling of the gastrointestinal tract comprises a capsule housing bounding a cavity, a sampling aperture formed in the capsule housing and providing fluid communication between the cavity and an exterior of the capsule housing, and a sampling hydrogel positioned inside the cavity. Upon exposure to a sample fluid, the sampling hydrogel is configured to absorb the sample fluid, expand within the cavity, and store the sample fluid for subsequent analysis. The device further comprises a sealing member positioned within the cavity between the sampling hydrogel and the sampling aperture. Expansion of the sampling hydrogel within the cavity presses the sealing member into engagement with the sampling aperture to seal the cavity. The device further comprises a biodegradable coating disposed within the sampling aperture. Degradation of the biodegradable coating exposes the sampling aperture to permit fluid flow into the cavity.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic illustration showing a capsule traveling through a GI tract, according to some embodiments;

FIG. 2A depicts one embodiment of a capsule at a first location in the GI tract;

FIG. 2B depicts the capsule of FIG. 2A at a second location in the GI tract;

FIG. 2C depicts the capsule of FIG. 2A at a third location in the GI tract;

FIG. 2D depicts the capsule of FIG. 2A at a fourth location in the GI tract;

FIG. 3 is a partially exploded view of a capsule, according to some embodiments;

FIG. 4A is a schematic illustration showing a first step of a method of manufacturing a coating for a capsule according to some embodiments using a drop casting technique;

FIG. 4B is a schematic illustration showing a second step of the method of FIG. 4A;

FIG. 4C is a schematic illustration showing a third step of the method of FIG. 4A;

FIG. 4D is a schematic illustration showing a fourth step of the method of FIG. 4A;

FIG. 4E is a schematic illustration showing a fifth step of the method of FIG. 4A;

FIG. 5 depicts an alternative embodiment of a capsule;

FIG. 6A is a plot showing the dissolution profiles of Eudragit L100-55 at various pH levels;

FIG. 6B is a plot showing the dissolution profiles of Eudragit L100 at various pH levels;

FIG. 6C is a plot showing the dissolution profiles of Eudragit S100 at various pH levels;

FIG. 6D is a bar graph showing the dissolution percentages of biodegradable coating polymers after 2 hours of exposure to various pH levels;

FIG. 7A is a plot showing the dissolution profiles of Eudragit EPO at various pH levels;

FIG. 7B is a plot showing the dissolution profiles of Chitosan at various pH levels;

FIG. 7C is a bar graph showing the dissolution percentages of biodegradable coating polymers after 1 hour of exposure to various pH levels;

FIG. 8 is a bar graph showing cytotoxicity of biodegradable coatings before and after NIR drying;

FIG. 9A is a plot showing spectroscopy results for enteric polymers in powder form;

FIG. 9B is a plot showing spectroscopy results for enteric polymer films;

FIG. 10A is a plot showing a thermogravimetric analysis of various forms of Eudragit L100-55;

FIG. 10B is a plot showing a thermogravimetric analysis of various forms of Eudragit EPO;

FIG. 11A is a microscopic image of a double layer biodegradable coating dried in the ambient environment;

FIG. 11B is a microscopic image of the double layer biodegradable coating of FIG. 11A after 2 hours of exposure to a pH of 1.2;

FIG. 11C is a microscopic image of the double layer biodegradable coating of FIG. 11B after 6 hours of exposure to a pH of 6.8;

FIG. 11D is a microscopic image of the double layer biodegradable coating of FIG. 11C after 1 hour of exposure to a pH of 5.5;

FIG. 11E is a microscopic image of a double layer biodegradable coating dried using NIR;

FIG. 11F is a microscopic image of the double layer biodegradable coating of FIG. 11E after 2 hours of exposure to a pH of 1.2;

FIG. 11G is a microscopic image of the double layer biodegradable coating of FIG. 11F after 6 hours of exposure to a pH of 6.8;

FIG. 11H is a microscopic image of the double layer biodegradable coating of FIG. 11G after 1 hour of exposure to a pH of 5.5;

FIG. 12A is an SEM image of a double layer biodegradable coating dried in the ambient environment;

FIG. 12B is an SEM image of a double layer biodegradable coating dried using NIR;

FIG. 13A is a bar graph showing a microbiota composition of the GI tract and comparison to a capsule administered to a first animal; and

FIG. 13B is a bar graph showing a microbiota composition of the GI tract and comparison to a capsule administered to a second animal.

### DETAILED DESCRIPTION

At least some aspects described herein are directed to targeted sampling of microbial communities, proteins, chemicals and other biomarkers throughout the GI tract.

Human microbiome sampling is becoming an essential aspect of understanding the mechanisms of microbiota-drug interactions as well as the degree to which this complex interplay can affect the drug efficacy and bioavailability. Much of what is known regarding the structure and function of the human gut microbiome has been ascertained from ex-situ culturing and/or sequencing of bacteria from fecal samples. However, only a small fraction of gut bacteria is available and culturable from fecal samples, and thus efforts have been made to develop tools that will enable direct sampling of microorganisms from the gastro-intestinal (GI) tract. For example, colonoscopy and/or gastroscopy methods are currently used, but these methods are limited to sampling at certain sections throughout the GI tract, and are invasive approaches which cause patient discomfort and can lead to decreased compliance. Other approaches have used smart functional capsules with the ability to collect samples at different targeted locations in the GI tract, and these methods can address several limitations associated with conventional colonoscopy and gastroscopy. Furthermore, capsule-based devices can improve patient comfort, without the requirement of being administered in clinical settings. For instance, the PillCam™ capsule endoscopy (CE) technology is used for collecting images from hard-to-reach areas throughout the GI tract to diagnose diseases related to the small intestine, such as obscure GI bleeding, tumors, Crohn's disease, angiodysplasia, celiac disease, and polyposis. However, this technology lacks the ability to collect and store samples as it travels through the GI tract.

Efforts in developing new capsules with different methods for sampling the gut microbiome can be classified into two main categories: active and passive devices. In active devices, an actuation and sampling mechanism are often attained by using an on-board battery that provides the required energy to actuate various plungers, pistons, biopsy forceps, etc., which collect and store samples within the capsule. However, in such devices, the battery often occupies a large fraction of the capsule volume, which may limit the space in which samples can be stored, and active devices typically exhibit a high risk of failure and possibility of leakage of caustic electrolytes that can cause severe corrosive injury and liquefactive necrosis. To avoid these and other drawbacks associated with active devices, various passive actuation sampling mechanism approaches have been exploited, thereby enabling the capsules to be more compact and economically viable, with fewer safety-associated issues. In such designs, the capsule moves through the GI tract via peristalsis motion with an average speed of 1-2 cm/min and the samples are collected through simple passive actuations such as capillary wicking actions or pressure differentials forces. However, the inventors have recognized and appreciated that there are numerous important design considerations that have not been addressed by existing passive devices. For example, some existing approaches rely on capsule assemblies requiring a well-sealed vacuum chamber inside a capsule, which significantly increases the complexity of the device. Other approaches have relied on retrieval of a sampling device via a string extending upstream from the GI tract, which can cause discomfort for a patient.

In some embodiments, a capsule with an aperture that forms an opening from the exterior of the capsule to the interior is provided. The aperture may be covered with a pH-sensitive enteric polymer coating that dissolves when it enters the basic environment of the small intestine. In some embodiments, inside the capsule is a hydrogel that draws a fluid sample into the capsule. As fluid enters the capsule, the hydrogel swells. An elastomeric disk may be provided with the hydrogel. When the hydrogel swells, the elastomeric disk may be pushed against the aperture to seal the capsule from the inside, thereby preventing any further fluid exchange.

The inventors have recognized that, in some cases, disposing a biodegradable coating on the exterior of the capsule to cover the opening may require the capsule to have corners or collars in the vicinity of the aperture to contain the liquid solution during application.

The inventors have thus appreciated that, in some embodiments, it may be beneficial to dispose the biodegradable coating within the aperture rather than on the exterior of the capsule. Disposing the biodegradable coating within the aperture may allow the exterior of the capsule to take on a wider range of geometries, including smoother and more rounded geometries that may be better suited to passing through the GI tract. For example, a capsule may be an elongated oval shape with only a small aperture formed at one end. A biodegradable coating may be disposed only within the aperture, allowing the exterior of the capsule to have only rounded features. Alternatively or in addition, disposing the biodegradable coating within the aperture may, in some embodiments, increase ease of manufacturing. For example, in some embodiments, creating a coating layer by depositing coating material within a contained volume of the aperture may be easier than applying the coating material around the exterior surface of a rounded capsule. In some embodiments, depositing coating material within a contained volume of the aperture may facilitate control of the coating layer thickness. This may be especially beneficial when multiple coating layers are applied, as will be discussed below.

Another limitation of current designs is that they only target the small intestine. The biodegradable coating must resist degradation in acidic environments in order to survive the stomach, but must dissolve in basic environments in order to allow sampling in the basic environment of the small intestine. Because the large intestine is more acidic, no single pH-sensitive coating has been identified to withstand both the acidic environment of the stomach and the basic environment of the small intestine to reach the colon intact and dissolve therein. This limitation of enteric polymer coating materials has inhibited the development of a similar passive sampling device for the colon. As described above, currently available techniques for colon sampling are generally invasive, expensive, time-consuming, and require skilled technicians. Therefore, there remains an unmet demand for user-friendly targeted passive sampling from the colon.

The inventors have recognized that, in some embodiments, it may be beneficial to include multiple layers of biodegradable coating, where each layer dissolves at a different pH level. Such an arrangement may permit the capsule to target different areas of the body for passive sampling. For example, to target passive sampling from the colon, an outer layer may dissolve in a basic environment and an inner layer may dissolve in an acidic environment. The outer layer may survive through the stomach and protect the inner layer from the acidic environment therein. The outer layer may dissolve in basic environment of the small intestine, thereby exposing the inner layer. The inner layer may survive the small intestine to dissolve in the acidic environment of the colon, thereby allowing the passive sampling process to occur within the colon.

The inventors have recognized that, in some embodiments, it may be useful to use a drop casting process to form one or more of the biodegradable coatings. The inventors have appreciated that drop casting can be used to produce thick layers in a shorter period of time while maintaining control and simplicity in the application process. For example, an enteric polymer may be dissolved in a liquid solvent to form a polymer solution. Droplets of the polymer solution having a controlled or known size may be deposited with a controlled momentum onto a surface or into the aperture, quickly creating a coating layer with a known thickness. It should be appreciated, however, that in other embodiments, other methods for forming the biodegradable coating(s) may be used.

The inventors have also recognized that, in some cases, when solvents that are used to create the polymer solution are toxic or are otherwise not biocompatible, the biocompatibility of the coating may be compromised if the solvent becomes trapped inside the polymer matrix. Additionally, a solvent may act as a plasticizer in a polymer matrix, thereby potentially reducing the rigidity of the coating layer and potentially increasing the likelihood of premature breakage of the seal. Therefore, the inventors have appreciated that, in some embodiments, it may be desirable to remove a solvent from a coating layer after deposition.

The inventors have recognized that, in some embodiments, it may be useful to use near infrared (NIR) spectroscopy methods to dry the polymer solution and to remove the solvents after deposition. In NIR processes, waves of a known and controlled wavelength are emitted at a sample material. While NIR is conventionally used as a method of identifying unknown materials, the ability to control the wavelengths emitted allows for wavelengths to be selected that may have a higher absorbance in the solvent than in the enteric polymer. A higher absorbance in the solvent may result in evaporation of the solvent, while a lower absorbance in the enteric polymer may result in the polymer film structure sustaining little or no damage. For example, an NIR process may allow the solvent to be evaporated out of the polymer matrix in a relatively short time, thereby removing the solvent before the polymer matrix is damaged. It should be appreciated, however, that in other embodiments, other methods for removing the solvent may be used, such as using heat.

Additionally, in designs with multiple layers of biodegradable coating, the ability of each layer to dissolve at the desired point in the GI tract may rely upon a separation or stratification of the different biodegradable coatings into distinct layers. When the different biodegradable coatings are allowed to intermix, the effect is similar to creating a single layer with a concentration gradient therethrough. The result is that the overall coating can partially dissolve at each portion of the GI tract, potentially compromising the capsule's ability to specifically target a particular point in the GI tract (for example, the colon). To prevent intermixing, each layer of coating may be dried or cured after deposition and before any subsequent layers are deposited.

In view of the above, the inventors have recognized that, in some embodiments, it may be beneficial to apply NIR to a coating layer before applying a subsequent layer. For example, NIR may be used to quickly dry and remove solvents from a first polymer coating layer after it is drop cast within an aperture of a sampling capsule prior to drop casting a second polymer coating layer over the first layer. NIR may then be used to quickly dry and remove solvents from the second layer, resulting in a biocompatible coating having multiple distinct layers that are structurally uncompromised. It should be appreciated, however, that in other embodiments, other methods for achieving solidification may be used, such as using heat.

In some embodiments, a passive sampling device may comprise a capsule that may be ingested by a patient such that the capsule travels through the GI tract. The capsule may include a capsule housing defining a cavity and an absorbent sampling hydrogel positioned within the cavity. The capsule housing may include a sampling aperture that permits fluid from the GI tract (e.g., fluid containing microorganisms or proteins such as calprotectin) to flow into the cavity, where it is absorbed by the sampling hydrogel. Upon absorbing the fluid, the sampling hydrogel may expand within the cavity and press a sealing member (e.g., a sealing membrane) positioned in the cavity between the aperture and the hydrogel material into engagement with the aperture to seal the aperture, thereby restricting subsequent fluid flow into or out of the capsule. In this manner, the devices disclosed herein may utilize the sampling hydrogel as a medium to store microbial samples within a capsule and also as a means for providing passive mechanical actuation to seal the capsule once sampling is completed. Additionally, the inventors have appreciated that after the capsule is sealed, the hydrated sampling hydrogel within the capsule may provide an ideal living environment with nutrients for the sampled bacteria to survive before retrieval of the capsule. Moreover, according to some aspects, the sealing of the capsule may aid in protecting collected samples within the capsule from harsh environments located within the GI tract, thereby preserving the bacterial samples stored in the sampling hydrogel samples for subsequent analysis.

In some embodiments, a capsule may include a biodegradable coating comprising a plurality of biodegradable coating layers disposed within the sampling aperture. For example, a biodegradable coating may be an enteric coating that is configured to dissolve at a desired target location along the length of the GI tract. In one embodiment, an outer biodegradable coating layer and an inner biodegradable coating layer are provided within the sampling aperture. Accordingly, the outer biodegradable coating layer may dissolve when the capsule reaches a location prior to a target location (referred to herein as a priming location), and the inner biodegradable coating layer may dissolve when the capsule reaches the target location. Dissolution of the inner biodegradable coating may allow gut fluids to enter into the capsule cavity through the aperture. In some embodiments, the biodegradable coating may be disposed only within the sampling aperture such that the biodegradable coating is constrained within the sampling aperture (and does not extend to an external surface of the capsule). It should be understood that a capsule according to the current disclosure may include any suitable biodegradable coatings, as would be appreciated by one of skill in the art. For example, suitable coating materials include, but are not limited to, pH-sensitive polymeric materials such as basic butylated methacrylate (EUDRAGIT EPO), poly methacrylic acid-co-ethyl acrylate (EUDRAGIT L 100-55), poly methacrylic acid-co-methyl methacrylate (EUDRAGIT L100), hydroxypropyl methylcellulose phthalate (HP-55), hypromellose phthalate (HPMCP), cellulose acetate phthalate (CAP), and polyvinyl acetate phthalate (PVAP).

In some embodiments, the biodegradable coating is formed using a solution casting technique. In such a technique, a polymer is dissolved in a solution and the solution is coated onto the cap. In some embodiments, the cap of the sampling capsules is placed on a silicone (e.g., PDMS) holder that blocks the aperture of the cap while the biodegradable coating is deposited onto the cap to prevent the biodegradable coating from passing through the aperture. The solution may have a high viscosity and may dry quickly. After the coating dries, the cap may be lifted off of the holder. The solution may separate easily from the silicone holder, thus allowing the coating the remain with the cap and covering the aperture of the cap. In some embodiments, the coating can be formed by drop casting which involves releasing large droplets with controlled sizes and momentum that spread and wet the surface upon impact, as described below. In some embodiments, a doctor blade may be used to create a film with a uniform thickness. Doctor blading, also known as knife coating or blade coating, involves running a blade over a surface (or moving the surface underneath the blade). A small gap between the blade and the substrate determines how much solution can get through as the blade passes, spreading the solution uniformly over the substrate.

In some embodiments, each layer of the biodegradable coating is formed using a drop casting technique. In such a technique, a biodegradable coating polymer is dissolved in a solvent and the resulting solution is deposited within the sampling aperture of the capsule. In some embodiments, a cap of the sampling capsule is placed on a silicone (e.g., PDMS) holder that blocks the aperture while a first solution containing a first biodegradable coating polymer is deposited into the aperture to prevent the first solution from passing through the aperture. Droplets of the first solution having a known size may be deposited with a controlled momentum into the aperture to form a first coating layer having a first thickness. In some embodiments, a second solution containing a second biodegradable coating polymer is deposited into the aperture. Droplets of the second solution having a known size may be deposited with a controlled momentum into the aperture to form a second coating layer having a second thickness.

In some embodiments, a doctor blade may be used to create a film with a uniform thickness. Doctor blading, also known as knife coating or blade coating, involves running a blade over a surface (or moving the surface underneath the blade). A small gap between the blade and the substrate determines how much solution can get through as the blade passes, spreading the solution uniformly over the substrate.

The thicknesses of the first and second layers may be the same or different. A combined thickness of the overall coating may be chosen to be sufficient to prevent premature breakage of the coating. Selective variation in the thickness of each layer may allow for targeted sampling at particular points along the GI tract. For example, a thicker layer of the second coating may cause the second coating to require more time to dissolve (i.e., a longer dissolution time). The layers may be designed to target a section of the colon for sampling by selecting a thickness for the second coating that results in a dissolution time which corresponds with a passage time through the targeted section.

After the coating dries, the cap may be lifted off of the holder. The coating may separate easily from the silicone holder, thus allowing the coating the remain undamaged within the aperture of the cap. In some embodiments, NIR may be used to accelerate and improve the drying process. During an NIR process, wavelengths may be selected for emissions that have a higher absorbance in the solvent than in the polymer in order to facilitate removal of the solvent and drying of the solution. In some embodiments, NIR may be used multiple times during the drop casting process. For example, NIR may be used a first time to dry the first solution prior to depositing the second solution, and a second time to dry the second solution.

As noted above, a capsule may include a sealing member that may be pressed into engagement with a sampling aperture of the capsule by the sampling hydrogel after the sampling hydrogel absorbs a gut fluid sample and expands within the cavity of the capsule. According to some aspects, the sealing member may be configured to provide a desired gas permeability between the cavity of a capsule and an exterior environment. For example, in some embodiments, the sealing member may be formed as a polydimethylsiloxane (PDMS) membrane, which may provide gas permeability to allow a natural gas exchange between the GI tract and the interior of the capsule (i.e., the cavity), which may aid in maintaining the natural metabolism of sampled bacteria and promote their survival after the capsule is sealed. Other suitable materials for the sealing member include, but are not limited to, polyvinyl chloride (PVC), thermoplastic polyurethanes (TPU), cylic olefin copolymer (COC), and perfluoropolyether (PFPE).

Depending on the particular embodiment, capsules described herein may be able to maintain live bacteria viable for subsequent analysis for an extended period of time after the capsule is sealed via the sealing member. For example, in some embodiments, the sampling hydrogel and/or sealing member may be constructed and arranged to maintain live bacteria in a sample fluid viable (i.e., keep the live bacteria alive) for at least 1 hour, at least 5 hours, at least 10 hours, at least 20 hours, or at least up to 24 hours or more before the sampling fluid is retrieved for analysis.

In some embodiments, the device may be used to collect proteins or other biomarkers to investigate a condition in the body. For example, in some embodiments, the device may be used to collect calprotectin in the GI tract to diagnose inflammatory bowel disease (IBD). Calprotectin is a protein released by neutrophils when there is inflammation in the GI tract.

After passing through the GI tract, the sealed capsule may be excreted by a patient and subsequently recovered to analyze the gut microbiome samples contained therein. According to some aspects, the capsule may be constructed and arranged to permit facile disassembly once recovered, thereby permitting easy recovery of the sampling hydrogel containing the samples. For example, in some embodiments, the capsule housing may be formed from two or more housing portions that may be removably secured to one another to permit access to the cavity of the capsule. In one exemplary embodiment, a capsule housing may be formed from two capsule portions that attach to one another via a threaded interface, which may allow the capsule to be easily disassembled after being retrieved through excretion such that the sampling hydrogel within the capsule may be removed for future culture and analysis of bacterial samples contained therein. Other suitable interfaces include, but are not limited to a snap fit interface, and a friction or interference fit interface.

According to some aspects, prior to assembly of the various components of a capsule housing, the components may be treated to provide a hydrophilic coating on the capsule housing. The inventors have recognized and appreciated that such treatments may aid in facilitating the flow of sample fluid through a sampling aperture and into the interior of the cavity where the sampling fluid may be absorbed by the hydrogel material contained therein. In particular, a hydrophilic coating on the surface of the capsule housing may aid in providing a continuous pull of fluid from the gut into the narrow sampling aperture on the capsule. Additionally, a hydrophilic coating on an interior surface of the sampling aperture may aid the adhesion of the biodegradable coating to the sampling aperture in addition to further contribute to the pull of fluid from the gut. For example, in some embodiments, a hydrophilic surface modification may be performed by activating the surfaces of the housing components using an air plasma treatment followed by submersion in a polyethylene glycol (PEG) solution.

The hydrogel materials disclosed herein may be composed of hydrophilic polymer networks capable of absorbing large quantities of water while maintaining their structure. These polymer networks are typically crosslinked via covalent bonds, hydrogen bonds, van der Waals interactions, and/or physical entanglements. The devices disclosed herein take advantage of both the absorption capacity, as well as the mechanical properties of hydrogels to provide non-invasive sampling devices which can passively extract and secure samples from targeted locations along the GI tract.

In some embodiments, the sampling hydrogel within a capsule may be synthesized from a combination of acrylic acid (AA) and acrylamide (AM) monomers. It should be understood that the current disclosure is not limited to any particular ratio of these monomers to form a hydrogel material. For example, suitable ratios of these monomers may include, but are not limited to 10% AA/90% AM, 30% AA/70% AM, 50% AA/50% AM, 70% AA/30% AM, or 90% AA/10% AM. As discussed in more detail below, a hydrogel material may be formed by mixing these monomers with deionized (DI) water as well as methylene bis-acrylamide (MBA) as a cross-linker and ammonium persulfate (AP) as an initiator. While certain hydrogel materials are described herein, it should be understood that other hydrogel materials may be suitable, such as hydrogels based on other acrylic polymers (e.g., combinations of acrylic acid, acrylamide, poly(N-isopropylacrylamide), and/or poly(N,N-diethylacrylamide)) and/or non-acrylic polymers. In some embodiments, hydrogels may be synthesized from a combination of acrylamide and N,N′-methylenebisacrylamide (MBA).

It should be appreciated that a capsule housing according to the current disclosure may be made from any suitable biocompatible material. For example, in some embodiments, the capsule housing may be formed from a biocompatible polymeric material, such as a methacrylate polymer. Other suitable materials include, but are not limited to, commercially available biocompatible polymers such as Dental LT Clear, MED625FLX, and MED610, and/or other polymeric materials treated with PEG to provide biocompatibility. Moreover, it should be understood that the current disclosure is not limited to any particular method to form the capsule housing. For example, some embodiments described in more detail below utilize capsule housings formed by a 3D printing process. Other suitable manufacturing methods may include, but are not limited to casting methods, molding methods (e.g., injection molding), or other methods as would be appreciated by one of ordinary skill in the art.

Depending on the particular embodiment, a capsule may have any suitable dimensions. For example, a cylindrical capsule may have a length of between about 9 mm and about 23 mm and a diameter between about 4.5 mm and about 10 mm. For example, in one embodiment, a capsule may have a diameter of about 9 mm and a length of about 15 mm, which is smaller than a standard 000 size gelatin capsule (which has dimensions of 9.97×26.14 mm). Moreover, a sampling aperture formed in the capsule housing may have a diameter selected based on a size of a sealing member contained within the capsule. For example, in some embodiments, the diameter of the sampling aperture may be selected to be at least 1 mm smaller than a diameter of the sealing member, which may aid in ensuring proper sealing of the capsule with the sealing member. In one exemplary embodiment, the sampling aperture may have a diameter of about 5 mm.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

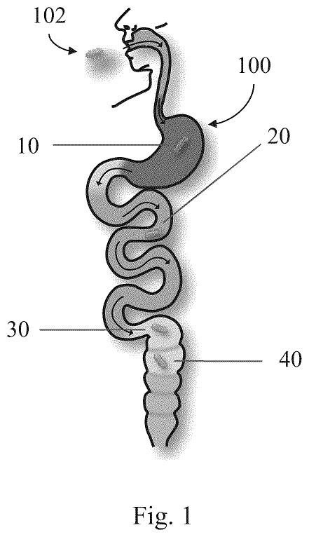

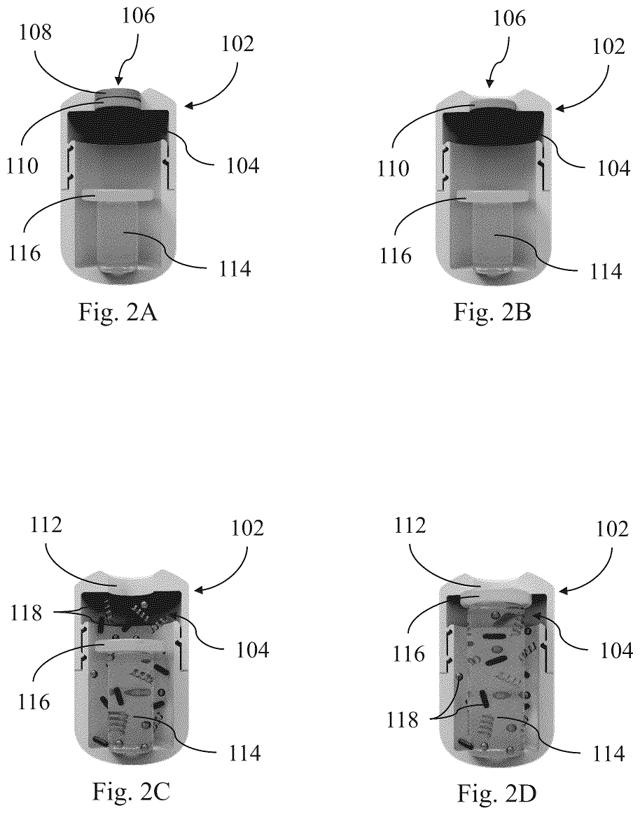

FIG. 1 is a schematic illustration of a sampling capsule 102 within a GI tract 100 at various locations 10, 20, 30, 40. FIGS. 2A, 2B, 2C, and 2D are schematic illustrations of the sampling capsule 102 in various states of operation that may correspond, respectively, to the locations 10, 20, 30, 40 of FIG. 1. As shown in FIG. 2A, the capsule 102 includes a biodegradable coating 106 (e.g., an enteric coating), which has an outer biodegradable coating layer 108 and an inner biodegradable coating layer 110. The biodegradable coating 106 is disposed within a sampling aperture 112 of the capsule 102, such that the biodegradable coating 106 seals the sampling aperture 112. In this manner, the biodegradable coating 106 protects the components within a cavity 104 of the capsule, including a sampling hydrogel 114 and a sealing member 116, and delays sampling until the capsule reaches a target location within the GI tract. In some embodiments, the biodegradable coating 106 may be disposed only within the sampling aperture and not on an external surface of the capsule. As illustrated in FIG. 2B, once the capsule reaches a priming location in the GI tract (which may be a point in the GI prior to the target location), the outer biodegradable coating layer 108 degrades and exposes the inner biodegradable coating layer 110 to GI fluids that surround the capsule. As illustrated in FIG. 2C, once the capsule 102 reaches the target location, the inner biodegradable coating layer 110 degrades and allows GI fluids, which contain sample material 118 (e.g., bacteria, proteins, chemicals, and/or potential other compounds of interest), to enter the capsule, where the fluids are absorbed by the sampling hydrogel 114. Upon absorbing the fluid, the sampling hydrogel swells and expands within the cavity 104 and fills substantially the entire volume of the cavity. Additionally, the expansion of the sampling hydrogel presses the sealing member 116 into contact with the sampling aperture, thereby sealing the sampling aperture via the mechanical force applied by the swollen hydrogel as shown in FIG. 2D. As illustrated, the fluid absorbed by the sampling hydrogel may contain sample material 118 from within the GI tract. After the capsule is excreted, the capsule may be disassembled to allow retrieval of the sampling hydrogel and the bacteria samples contained therein such that bacteria may be subject to future culture and analysis.

When a patient swallows an exemplary embodiment of a sampling device with two layers of biodegradable polymer coating, the device may be exposed to the saliva at pH 7 for typically less than 1 minute. As the device moves towards the stomach, the pH drops to roughly 3. The device may remain inactivated as the outer enteric polymer remains un-ionized in the acidic pH environment of the stomach. As the device moves further down the gastrointestinal tract, the pH increases when it reaches the small intestine with an average pH of 6.8 and, typically, a maximum pH of 7.5. As the pH exceeds the dissolution threshold of the outer biodegradable coating layer, the outer polymer starts to ionize, initiating the polymer dissolution. Once the outer biodegradable coating layer has dissolved, the inner biodegradable coating layer is exposed to the fluids of the small intestine. At this point, the device may remain inactivated as the inner biodegradable coating layer remains un-ionized in the basic pH environment of the small intestine. When the device reaches the ascending colon, where the pH suddenly drops again, the inner biodegradable coating layer may start to ionize and dissolve in the acidic environment of the colon. Once the inner biodegradable coating layer has dissolved, intestinal fluid may enter the cavity 104 and the hydrogel may start to swell by absorbing the intestinal fluid while pushing a sealing member inside the cavity towards the sampling aperture. With the device aperture sealed with the sealing member, the device can move along through the remainder of the GI tract with no fluid exchange.

In some cases, it may take approximately 24 to 72 hours for the device to travel through the colon. Depending on the intended application of the device, biodegradable coating dissolution and/or hydrogel elongation may be tuned to a desired target location in the colon. Biodegradable coating dissolution may be tuned by selecting an appropriate thickness of one or more coating layers as described above. For example, in some embodiments, the biodegradable coating may be configured to target the ascending colon by selecting a coating thickness for an inner biodegradable coating layer to produce total dissolution in two hours after entry of the device into the colon. Afterwards, the hydrogel may be configured to elongate in one hour, and finally afterwards the device may be configured to be sealed by the sealing member in 0.5 hours. In an example targeting the transverse colon, the biodegradable coating may be configured to target the ascending colon by selecting a coating thickness for an inner biodegradable coating layer to produce total dissolution in 11 hours after entry of the device into the colon Afterwards, the hydrogel may be configured to elongate in one hour, and finally afterwards the device may be configured to be sealed by the sealing member in 0.5 hours. These time target time intervals may be selected to allow for a safety margin of a desired length of time to ensure the device is sealed prior to excretion. After excretion, the capsule can be retrieved, disassembled, and the sample can be analyzed for further investigations.

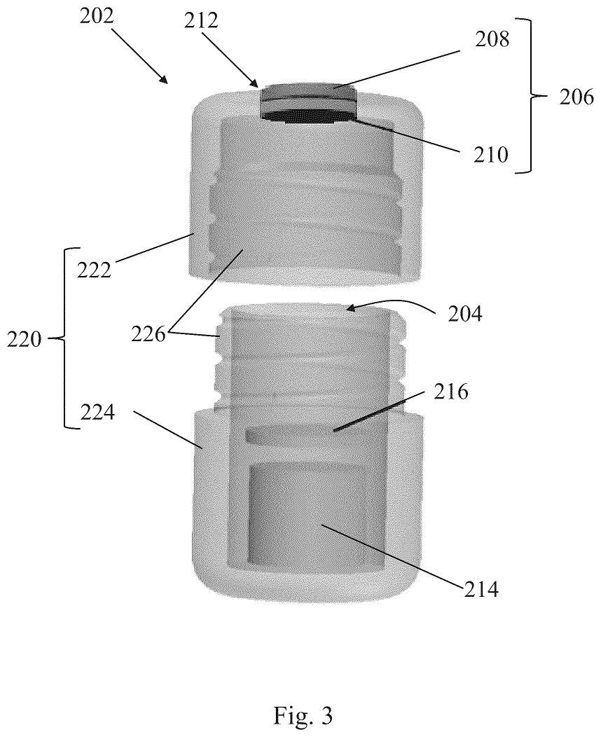

FIG. 3 is a schematic exploded view of one embodiment of a capsule 202 for passive sampling of the gastrointestinal tract. The capsule 202 includes a capsule housing 220 including a first housing portion 222 (also referred to herein as a cap) and a second housing portion 224. The first housing portion includes a sampling aperture 212 to provide fluid communication between an exterior of the capsule and a cavity 204 formed within the capsule housing 220. A biodegradable coating 206, which includes an outer biodegradable coating layer 208 and an inner biodegradable coating layer 210 is provided within the sampling aperture 212 to seal the sampling aperture 212 until the capsule 202 reaches a desired location within the GI tract. In some embodiments, the biodegradable coating may be disposed only within the sampling aperture such that the biodegradable coating 206 is constrained within the sampling aperture (and does not extend to an external surface of the capsule). A sampling hydrogel 214, and a sealing member 216 are positioned within the cavity 204, and as illustrated, the sealing member 216 is positioned between the sampling hydrogel 214 and the sampling aperture 212. As noted above, in some embodiments, the sealing member 216 may be a flexible membrane such as a PDMS membrane, and may provide for gas permeability between the cavity 204 and an exterior of the capsule 202 after the sealing member seals the cavity. Additionally, the first and second housing portions 222 and 224 include an attachment interface 226 to allow the capsule housing to be easily disassembled for retrieval of the hydrogel material after a sample has been collected. In the depicted embodiment, the attachment interface 226 comprises corresponding threaded features formed on the first and second housing portions that cooperate to form a screw interface. However, as noted above, other attachment arrangements such as a snap fit or interference fit may be suitable in other embodiments.

In the depicted embodiment, the sampling aperture has a circular shape. However, it should be appreciated that the sampling aperture 212 may have any suitable shape. The shape of the sampling aperture 212 may determine the shape of the biodegradable coating 206 disposed therein. For example, in some embodiments, the sampling aperture may be a square, rectangle, oval, or any other suitable shape.

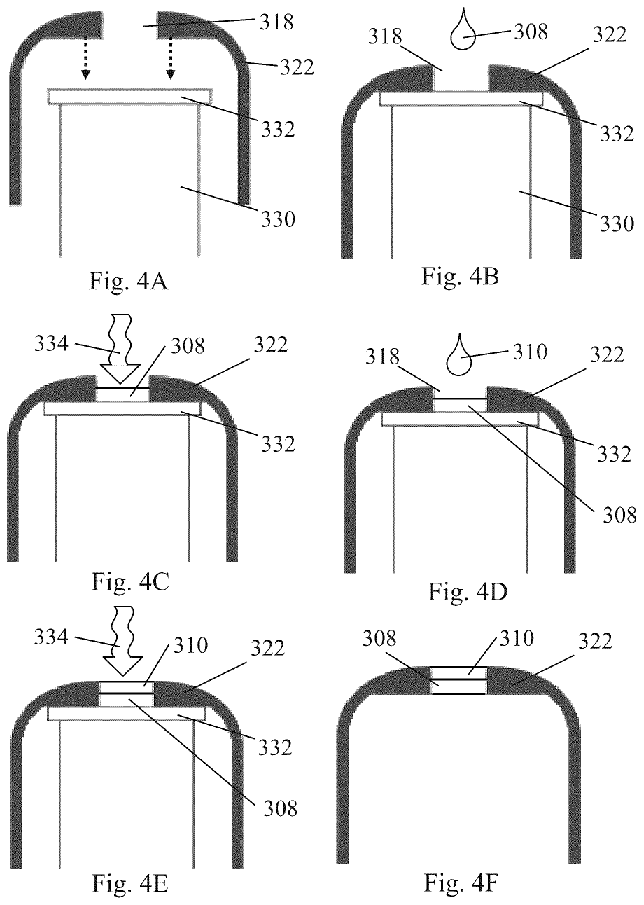

FIGS. 4A-4F show a schematic view of one embodiment of a method of manufacturing a capsule with multiple layers of a biodegradable coating disposed within a sampling aperture of the capsule. In FIG. 4A, a first housing portion 322 of a capsule is placed onto a fixture 330 in the direction of the arrows shown. The fixture 330 has a sealing plate 332 configured to provide a liquid-tight seal with the first housing portion 322 of the capsule at a perimeter of a sampling aperture 318 of the capsule. The sealing plate 332 may be a silicone material (e.g., PDMS) or any other material that provides a liquid-tight seal without bonding with or adhering to the biodegradable coatings. A liquid solution of a first biodegradable coating 308 is introduced into the aperture 318. The liquid-tight seal between the sealing plate 332 and the first housing portion 322 of the capsule may retain the first biodegradable coating 308 within the aperture, as shown in FIG. 4C. The first biodegradable coating 308 is then allowed to dry. In some embodiments, drying may include the use of NIR, as represented by arrow 334 in FIG. 4C. Any suitable parameters for the use of NIR may be selected as appropriate, including a wavelength, operating power, and scanning speed as discussed below.

In some embodiments, a wavelength used in an NIR drying process may be greater than or equal to 300 mm, 400 nm, 500 mm, and/or any other appropriate wavelength. In some embodiments, the wavelength may be less than or equal to 2.0 μm, 2.25 μm, 2.5 μm, and/or any other appropriate wavelength. Combinations of the foregoing are contemplated including, for example, greater than or equal to 300 mm and less than or equal to 2.5 μm, greater than or equal to 400 mm and less than or equal to 2.25 μm, and/or any other appropriate combination of the foregoing. Of course, while particular ranges for the NIR wavelength are provided above, it should be understood that other ranges both greater than and less than those noted above are also contemplated as the disclosure is not limited in this fashion.

In some embodiments, an operating power used in an NIR drying process may be greater than or equal to 1 kW, 3 KW, 5 KW, and/or any other appropriate power level. In some embodiments, the operating power may be less than or equal to 8 kW, 10 KW, 15 kW, and/or any other appropriate power level. Combinations of the foregoing are contemplated including, for example, greater than or equal to 1 kW and less than or equal to 15 kW, greater than or equal to 3 kW and less than or equal to 8 kW, and/or any other appropriate combination of the foregoing. Of course, while particular ranges for the NIR operating power are provided above, it should be understood that other ranges both greater than and less than those noted above are also contemplated as the disclosure is not limited in this fashion.

In some embodiments, a scanning speed used in an NIR drying process may be greater than or equal to 1 m/min, 3.6 m/min, 5 m/min, and/or any other appropriate speed. In some embodiments, the scanning speed may be less than or equal to 7.5 m/min, 10 m/min, 12.5 m/min, and/or any other appropriate speed. Combinations of the foregoing are contemplated including, for example, greater than or equal to 1 m/min and less than or equal to 12.5 m/min, greater than or equal to 3.6 m/min and less than or equal to 7.5 m/min, and/or any other appropriate combination of the foregoing. Of course, while particular ranges for the NIR scanning speed are provided above, it should be understood that other ranges both greater than and less than those noted above are also contemplated as the disclosure is not limited in this fashion.

Once the first biodegradable coating 308 has dried, a second biodegradable coating 310 may be introduced into the aperture 318, as shown in FIG. 4D. The second biodegradable coating 310 is then allowed to dry. In some embodiments, drying may include the use of NIR, as represented by arrow 334 in FIG. 4E. The first housing portion 322 is removed from the fixture, with the sealing plate 332 being separated from the first biodegradable coating 308 without damaging the coating. In some embodiments, the method described above may produce a biodegradable coating that is disposed only within the sampling aperture such that the biodegradable coating is constrained within the sampling aperture (and does not extend to an external surface of the capsule).

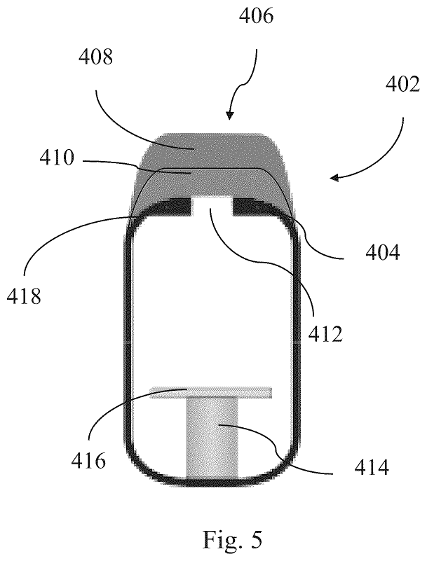

FIG. 5 shows a schematic illustration of an alternative embodiment of a capsule 402. The capsule 402 includes a biodegradable coating 406 (e.g., an enteric coating), which has an outer biodegradable coating layer 408 and an inner biodegradable coating layer 410. The biodegradable coating 406 is disposed on an external surface 418 of the capsule 402 and across a sampling aperture 412 of the capsule 402, such that the biodegradable coating 406 seals the sampling aperture 412. In this manner, the biodegradable coating 406 protects the components within a cavity 404 of the capsule, including a sampling hydrogel 414 and a sealing member 416, and delays sampling until the capsule reaches a target location within the GI tract. Once the capsule reaches a priming location in the GI tract (which may be a point in the GI prior to the target location), the outer biodegradable coating layer 408 degrades and exposes the inner biodegradable coating layer 410 to GI fluids that surround the capsule. Once the capsule 402 reaches the target location, the inner biodegradable coating layer 410 degrades and allows GI fluids, which contain bacteria, to enter the capsule, where the fluids are absorbed by the sampling hydrogel 414. Upon absorbing the fluid, the sampling hydrogel swells and expands within the cavity 404 and fills substantially the entire volume of the cavity. Additionally, the expansion of the sampling hydrogel presses the sealing member 416 into contact with the sampling aperture, thereby sealing the sampling aperture via the mechanical force applied by the swollen hydrogel. The fluid absorbed by the sampling hydrogel may contain bacteria from within the GI tract. After the capsule is excreted, the capsule may be disassembled to allow retrieval of the sampling hydrogel and the bacteria samples contained therein such that bacteria may be subject to future culture and analysis.

In the depicted embodiment, the biodegradable coating 406 initially covers or surrounds only a portion of the capsule 402. However, it should be appreciated that other arrangements may be suitable, such as arrangements in which the biodegradable coating 406 covers or surrounds the entire exterior of the capsule 402. In some embodiments, the biodegradable coating may have a curved outer surface. In some embodiments, the biodegradable coating may have a flat outer surface. The biodegradable coating may be any shape that seals the sampling aperture of the capsule. The biodegradable coating of FIG. 5 may be deposited on the capsule by any appropriate method, including drop casting, doctor blading, solution casting, or spray coating. It should be noted that any of these methods may produce a biodegradable coating that may be disposed only within the sampling aperture such that the biodegradable coating is constrained within the sampling aperture (and does not extend to an external surface of the capsule).

### EXAMPLES

### Device Fabrication

In one example, a device for passive sampling of the colon was manufactured using 3D printing. The device consisted of four components: a biodegradable coating including a first and second biodegradable coating layer, a 3D-printed housing including a first housing portion and a cap, a sampling hydrogel, and a gas permeable PDMS membrane. The 3D-printed housing was designed with SolidWorks (Dassault Systèmes) and printed using a Form 3 3D printer using a stereolithography technique followed by pure isopropyl alcohol (IPA) rinsing and UV photocuring for 15 minutes at 60° C. The PDMS membrane was fabricated using a standard 1:10 ratio of curing agent to silicone base and cured at 70° C. for 4 hours. A computer-controlled CO₂ laser cutting and engraving system (PLS6MW from Universal Laser, Inc., operating at a wavelength of 10.6 μm) was utilized to cut a circle having a diameter of 6 mm in the PDMS. The hydrogel was synthesized by dissolving acrylamide and MBA crosslinker in water. The solution was then degassed by bubbling nitrogen gas for 10 minutes and then adding ammonium persulfate (APS) to the solution to act as an initiator. The solution was finally poured into a mold and polymerized overnight at 70° C. To prevent the device from floating in a fluid, a water-jetted cylindrical copper disk was placed at the bottom of the housing. The copper disk was coated with PDMS to preserve the biocompatibility of the device.

### Characterization of Biodegradable Coating Layers

pH-sensitive polymers include the broad categories of polyacids and polybases. Polyacids can accept protons at low pH and release protons either at neutral pH or higher pH. Polyacids such as Eudragit L100, Eudragit L100-55, and Eudragit S100 can therefore be dissolved in aqueous media at high pH but are insoluble in lower pH ranges. Polybases can accept protons at higher pH and release protons either at neutral pH or lower pH ranges. Polybases such as Chitosan and Eudragit EPO can therefore be dissolved in aqueous media at low pH but are insoluble at higher pH ranges. The release and acceptance of protons, as well as the resulting solubility or insolubility of these polyelectrolytes, are controlled by the pH in the surrounding environment.

To better evaluate dissolution of the various pH-sensitive polymers, methylene blue and deep orange food coloring dye were added to solutions of Eudragit L100-55 and Eudragit EPO, respectively. These dyes allowed for peak absorbance in the polymers during ultraviolet visible spectrophotometry (UV-Vis). The solutions were then casted on acrylic sheets using doctor blading to obtain uniform film thicknesses. After casting each layer, to avoid any solvent entrapment in the film composition, the film was fully dried using NIR (Adphos NIR-126-250 Modul with 3.6 m·min⁻¹ line speed of the conveyer and 3 kW lamp power). Once the layers were dry, identical disks with diameters of 10 mm were cut using the computer-controlled CO₂ laser cutting and engraving system with a wavelength of 10.6 μm.

Dissolution testing of the pH-sensitive polymer samples was carried out in 900 mL of various dissolution media at 37° C. using USP Type II apparatus (PTWS instrument, Pharma Test, Hainburg, Germany) at a paddle speed of 100 rpm. The dissolution media had pH values of 1.2, 3, 5.5, 6.8, and 7.4. Polymer samples were withdrawn from the dissolution media at predetermined time points and analyzed by UV-Vis using a BMG Clariostar microplate reader (BMG Labtech, Germany) before being returned to the dissolution media. The released dye percentage from the formulations was measured using UV-Vis techniques at a wavelength corresponding to the peak wavelength of the appropriate dye. All tests were performed in triplicate.

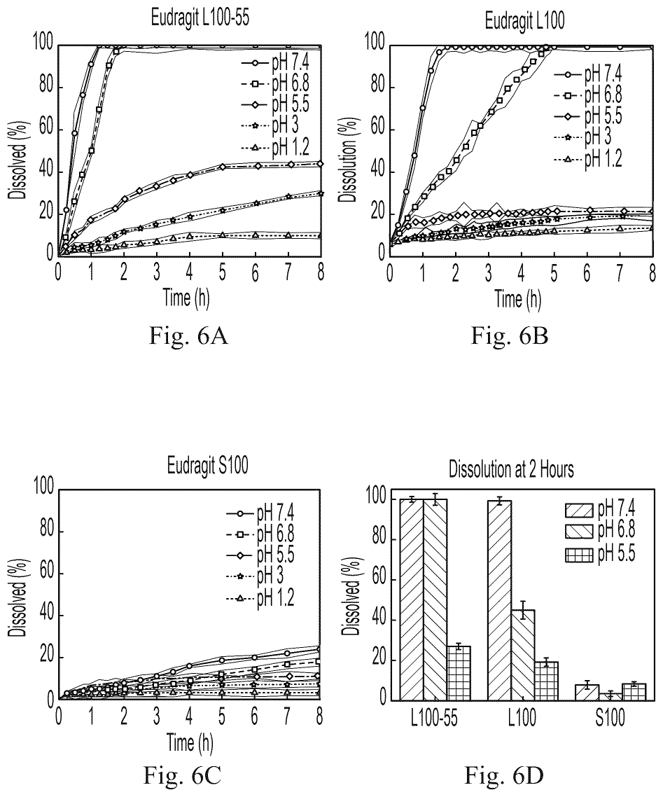

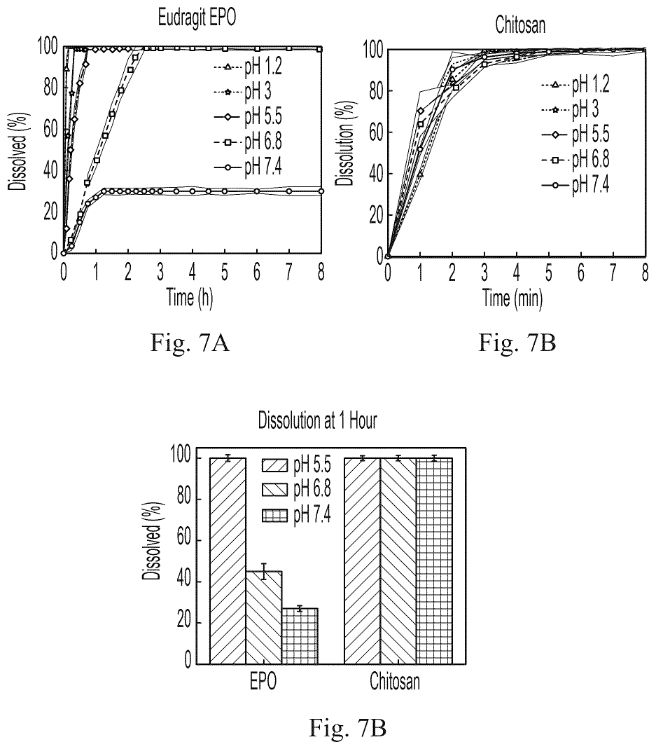

The obtained dissolution tests of different polyacids (FIGS. 6A-6D) and polybases (FIGS. 7A-7C) under GI tract pH environments, show a diverse variety of dissolution profiles suggesting that various dissolution kinetics and profiles may be achieved by different formulations.

Among polyacid polymers in a simulated fasting state of the stomach (pH=1.2), as shown in FIGS. 6A-6C, all three polyacids showed minimal dissolution (<10%) during the simulated gastric residence time (2 hours). Continuing the experiment until 8 hours of residence time did not result in substantial changes to this trend. In a simulated fed state (pH=3.0), also shown in FIGS. 6A-6C, all three polyacids showed below 15% dissolution within 2 hours of gastric residence time. However, Eudragit L100-55 showed a slow continuous dissolution trend that reached ˜30% after 8 hours of residence time (FIG. 6A).

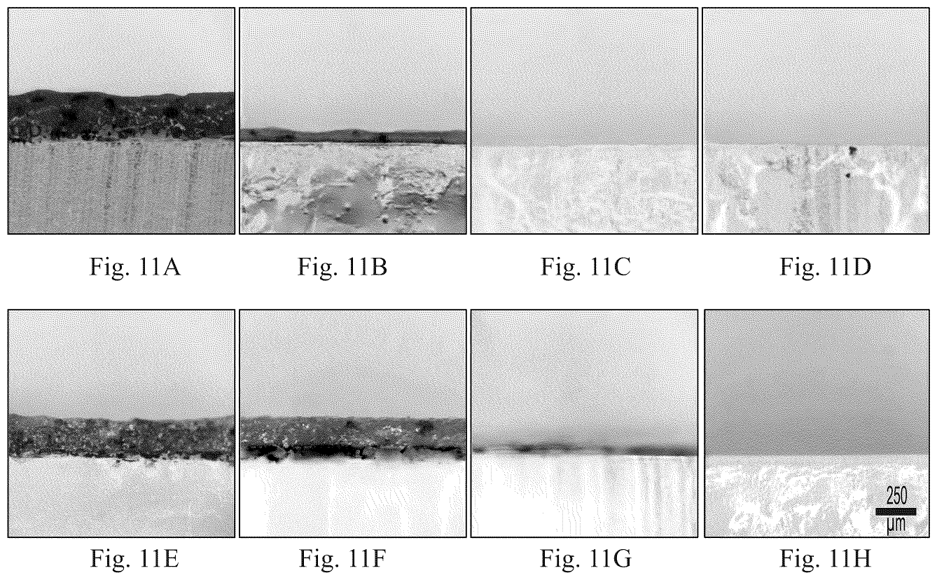

At pH=5.5, which generally simulates the pH of both duodenum and cecum, Eudragit L100-55 shows ˜30% dissolution after 2 hours (FIG. 6A). However, Eudragit L100 and Eudragit S100 remained mostly insoluble after 8 hours, showing 21% (FIG. 6B) and 11% (FIG. 6C) dissolution, respectively. When pH=6.8, generally simulating the proximal small intestine region, the threshold pH level for dissolution of Eudragit L100-55 is met. This may have caused Eudragit L100-55 to become fully ionized and fully dissolved within 2 hours (FIG. 6A). The complete dissolution of Eudragit L100 occurred after 5 hours (FIG. 6B), and Eudragit S100 only dissolved by 20% after 8 hours (FIG. 6C).

Dissolution profiles for the polybase polymers Eudragit EPO and Chitosan are shown in FIGS. 7A-7C. Chitosan showed relatively rapid dissolution at all pH levels, with complete dissolution occurring at each pH level within 5-6 hours. Eudragit EPO showed complete dissolution within 1 hour at pH levels of 1.2, 3.0, and 5.5, and complete dissolution within 3 hours at a pH of 6.8. At a pH of 7.4, however, dissolution of Eudragit EPO did not exceed ˜30% for the entire 8 hours of residence time.

### Near Infrared Drying

In some studies, NIR technology was applied to increase the evaporation rate of the solvents and to prevent intermixing of the polymer layers while preserving the integrity of the polymer film structure.

Characterization of the Reduced Cytotoxicity from NIR

In-vitro cytotoxicity of samples having double-layer biodegradable coatings was determined by MTT (tetrazolium bromide) assay using human mesenchymal stem cells (hMSCs). Cytotoxicity was investigated both before and after NIR processing. The cytotoxicity of a capsule with no biodegradable coating was selected as a control, in part to ensure that the cured 3D-printed resin is non-toxic to the cells. Examinations of the cytotoxicity of the biodegradable coating were performed because the coating comprised two layers that were drop casted. The examinations were performed to verify whether the drop casted layers released organic solvents as potential carcinogens and neurotoxins.

Cells were cultured in Dulbecco's Modified Eagle Medium (DMEM) with 10% fetal bovine serum (FBS), 100 units/mL penicillin, and 100 units/mL streptomycin. Cells were grown in a humidified incubator at 37° C., 5% CO₂, and 95% relative humidity. Once the cells were confluent (˜80%), samples were transferred to a 24-well plate and UV sterilized for 2 hours (1 hour per side), then cells were seeded at a density of 2×10⁴ cells per well and a final volume of 2 mL. After incubation for 24 hours, the culture medium in each well was replaced with 1800 μL of fresh DMEM and 200 μL of an MTT solution (5 mg/mL thiazolyl blue in PBS) and incubated for another 1 hour. The optical density (OD) value of the mixture was measured at 490 nm using the BMG microplate reader. Cell viability was calculated using the following equation:

Cell viability percentage=live cells on test surface/live cells on control surface×100%

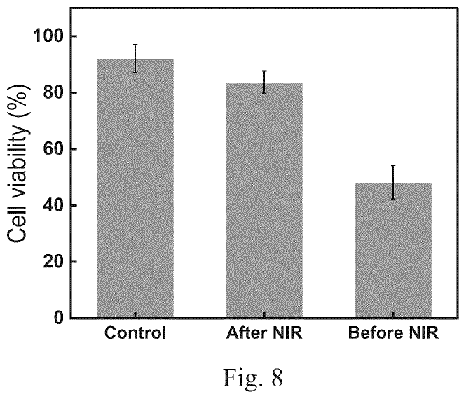

As shown in FIG. 8, the cell viability of the control was calculated as 92%, indicating that the capsule housing was biologically safe. However, the cell viability of the drop casted films that were dried in the environment showed a drop to <50% before applying NIR. This toxicity of the double-layer film may be primarily driven by the top layer, as a greater proportion of solvent may have been trapped inside the structure of the Eudragit L100-55 as a result of its higher molecular weight as compared to the bottom layer with lower molecular weight, as will be discussed in greater detail below.

Cell viability after NIR heat treatment increased to 84%, suggesting that the polymer dissolution is harmless to the cells. The biocompatibility of the NIR-treated biodegradable coating was due to the evaporation of trapped solvents. This is consistent with the thermogravimetric analysis (TGA) results discussed below.

### ATR-FTIR Characterization of NIR Effects

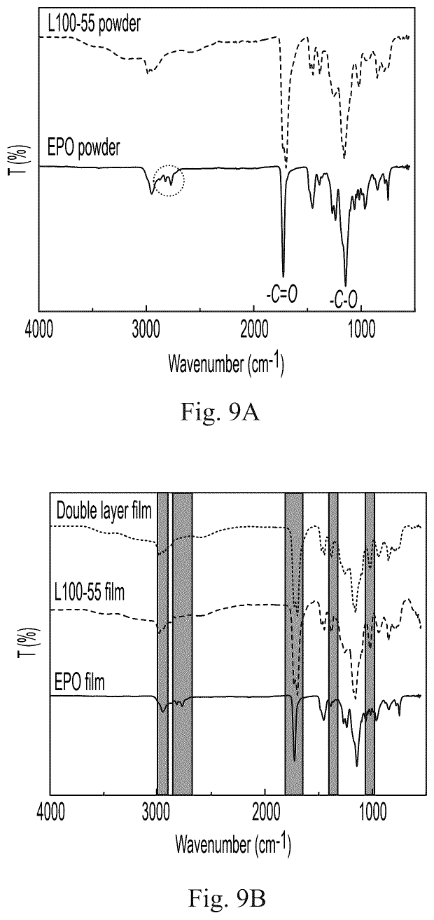

To ensure that the double-layer drop casted films showed no intermixing effects after using NIR processing, infrared spectra of single layers of L100-55 and EPO polymers were compared with infrared spectra of the double layer film by means of attenuated total reflection Fourier transform infrared spectroscopy (ATR-FTIR) using a PerkinElmer Spectrum 100 FTIR Spectrometer over the range of 4000 cm⁻¹ to 550 cm⁻¹ by making 50 scans. Single layers of Eudragit L100-55 and Eudragit EPO were evaluated both in film states and in powder states. FIG. 9A shows the comparative ATR-FTIR spectra of pure EPO and L100-55 powders, and FIG. 9B shows the comparative ATR-FTIR spectra of pure EPO and L100-55 films as well as the double layer coating film.

As shown in FIG. 9A, the ATR-FTIR spectra for pure L100-55 and EPO powders demonstrated predominant characteristic peaks at about 1700 cm⁻¹ and 1100 cm⁻¹ assigned to the C═O stretching vibration patterns of ester group and C—O bonds of carboxylic ester, respectively. Additionally, the spectrum for Eudragit EPO showed a stretching band at about 2850 cm⁻¹. This band may be for a dimethylamine group which may distinguish the L100-55 from the EPO.

As shown in FIG. 9B, the ATR-FTIR spectra of the films exhibited the important peaks for both L100-55 and EPO without any significant additional vibration bands for solvent residues. These observations revealed that the NIR processing is a capable drying technique that may remove all the solvents without affecting the polymer film structures.

Also shown in FIG. 9B, the ATR-FTIR spectrum for the double layer coating film demonstrated the characteristic peaks for a pure L100-55 polymer without any noticeable peaks that would indicate intermixing of the EPO layer into the L100-55 film. Since the top coating was a pure L100-55 film, this observation confirms that the expected separation of two polymer films may have originated from immiscible behavior of the initial polymer solutions.

### TGA Characterization of NIR Effects

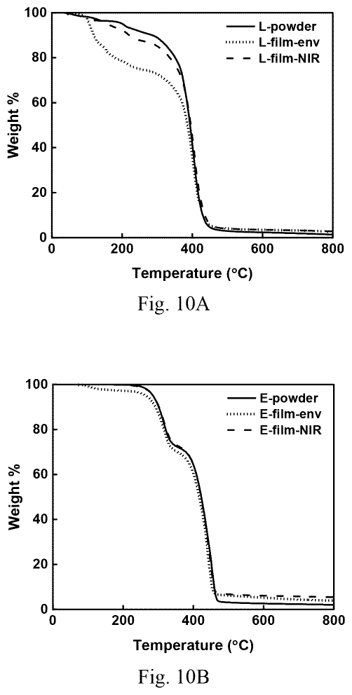

Thermal characterization of Eudragit L100-55 (shown in FIG. 10A) and Eudragit EPO (FIG. 10B) in both powder and film configurations was performed using thermogravimetric analysis (TGA). In one film configuration, the film was allowed to dry in the environment. In the other film configuration, the film was dried using NIR. No solvents were used in the powder configuration.

The differences in weight loss between the powder and film configurations may be due to the loss of moisture and solvents present in the film configuration, as these factors are absent from the powder configuration. Furthermore, films dried in the environment may trap a higher proportion of solvents than films dried using NIR. As shown in both FIGS. 10A and 10B, NIR drying may have removed a greater proportion of entrapped solvents than environmental drying, resulting in a TGA curve that is closer to that of the powdered form.

In all instances of Eudragit L100-55 (FIG. 10A), the weight loss from 50-150° C. may be attributable to the evaporation of excess solvent, while the loss from 225-425° C. may be due to thermal degradation of the polymer structure. For Eudragit EPO in FIG. 10B, the loss from 275-325° C. may be the result of solvent evaporation, while the larger drop from 375-450° C. may be thermal degradation.

NIR shows a significantly greater effect on Eudragit L100-55 than on Eudragit EPO. This may be due to the higher molecular weight of Eudragit L100-55. Eudragit L100-55, with a molecular weight of 320,000 g/mol, is considerably heavier and more viscous than Eudragit EPO, whose molecular weight is only 47,000 g/mol. This higher molecular weight may cause substantially more entanglement and entrapment of solvent particles, potentially making the solvent particles more difficult to remove without the aid of drying techniques such as NIR. As a result, the difference between Eudragit L100-55 dried by NIR and Eudragit L100-55 dried in the ambient environment was observed to be significantly greater than the difference between Eudragit EPO dried by NIR and Eudragit EPO dried in the ambient environment. Because the radiation from NIR is not destructive to the polymer structures, the solvent molecules may be targeted for evaporation without affecting the film structure or the capsule body.

### Microscopic Characterization of NIR Effects

To visualize the effect of NIR on the morphology and intermixing of the double layer biodegradable coating, cross-sectional images were taken of the double layer coating. Since the solvents of both Eudragit L100-55 and Eudragit EPO were substantially the same, there may have been a high chance of the polymers intermixing if the layers were slowly dried under environmental conditions. In some cases, intermixed polymers may produce unintended dissolution profiles at various pH levels. For instance, FIG. 11A shows the double layer formation when the top layer and bottom layers were dried in the ambient environment. The mixture shown in FIG. 11A confirms that two polymers are blended with each other. In this configuration, the double layer coating may not exhibit the intended response to various pH levels. Blending of the layers may have occurred in part because the slow drying process of the top layer may have allowed the solvents to dissolve the bottom layer. This effect, wherein application of the top layer may cause dissolution of the bottom layer over time, may result in intermixing of the polymers.

FIG. 11B shows the configuration of FIG. 11A after 2 hours of exposure to a pH level of 1.2. Under these conditions, the top layer may have been unable to protect the bottom layer from dissolution as a result of the intermixing of the two layers. Therefore, the orange layer dissolved completely and the thickness of the blue layer decreased significantly. FIG. 11C shows that, after 6 hours at pH 6.8, the remaining top layer was fully dissolved. This dissolution of the top layer should be expected due to the pH level exceeding the pH dissolution threshold of the top layer polymer (Eudragit L100-55). FIG. 11D shows simply that because the bottom layer had already dissolved as a result of the intermixing, there was no bottom layer left to dissolve at pH level 5.5.

FIGS. 11E-11H show the effects of NIR processing on the double layer coating. NIR processing was utilized to prevent blending of the polymers via rapid and effective drying of the films. As shown in FIG. 11E, the bottom and top layers were more distinctly separated and better stratified into individual layers. The layers showed virtually no dissolution or alteration in thickness after being submerged in pH 1.2 for 2 hours (FIG. 11F). This may be because the pH has not exceeded the dissolution threshold of the top layer of Eudragit L100-55. The top layer may therefore have been preserved and may have acted as a shield to prevent the bottom layer from being exposed to the lower-pH environment that would otherwise have caused the bottom layer to dissolve. However, when the pH rises to 6.8, as shown in FIG. 11G, the dissolution threshold of the top layer is met. FIG. 11G shows full dissolution of the top layer after 6 hours at a pH level of 6.8. Once the top layer had dissolved, the bottom layer was exposed to the higher pH environment. After the pH was then reduced to 5.5 for 1 hour, the bottom layer was perfectly ionized and dissolved as shown in FIG. 11H.

### SEM Characterization of NIR Effects

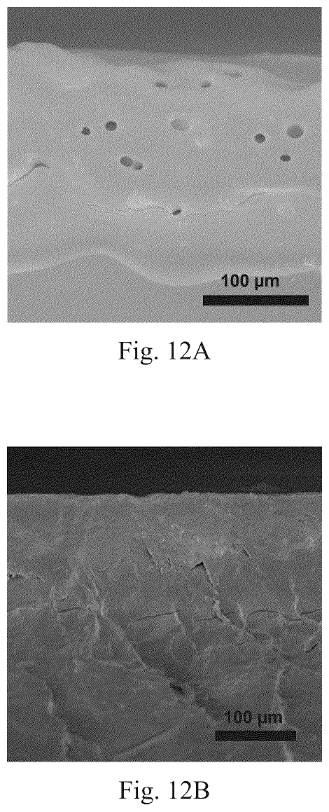

Additionally, high magnification cross-sectional images of the double layer biodegradable coating, both before and after NIR processing, are shown in FIGS. 12A and 12B. These images were taken using a scanning electron microscope (SEM) (Hitachi-S 4800, Tokyo, Japan). As seen in FIG. 12A, the top layer (Eudragit L100-55, dried in the ambient environment) showed significant porosity. The bottom layer (Eudragit EPO, dried in the ambient environment) shows significantly less porosity than L100-55. These pores may be formed as a result of the slow drying process in ambient environmental conditions. Additionally, and as described above, the higher molecular weight of Eudragit L100-55 may cause a greater degree of entanglement between the polymer chains and the particles of both solvent and air, effectively preventing the solvent particles and air particles from easily escaping the polymer structure.

FIG. 12B shows no similar porosity issues in the Eudragit L100-55 top layer when NIR was used to dry the coating layers. This may be a result of the pulse heat generated by the lamp energizing the solvent particles and air particles, thereby compelling the air bubbles to escape the polymer matrix and ultimately leading to a polymer structure that is free of pores and free of cracks. Briefly, the microscopic and SEM cross-sectional images after NIR processing may demonstrate the efficiency of this technique to not only achieve proper dissolution and protection at different pH environments, but also to assist with acceptable polymer morphology.

### Bacterial Sampling

### In-Vitro Study

To assess the sampling capabilities of both the hydrogel and the overall device, four containers were prepared containing mixtures of gut microbiota. Each container contained a different mixture, as shown in Table 1 below. The composition of a sample drawn by each device and each hydrogel was compared to the composition of the initial mixture. The bacteria used included Lactobacillus cremoris (Firmicutes), Bacteroides fragilis (Bacteroidetes), Escherichia coli LF82 (Proteobacteria), and Akkermansia muciniphila (Verrucomicrobia), as shown in Table 2. The bacterial strains were cultured inside an anerobic chamber at 37° C. overnight with the respective broth, also shown in Table 2.

TABLE 1

| Con- | | | | | |
|---|---|---|---|---|---|
| tainer | Strain(s) | Pre | Post | Capsule | Gel |
| M1 | Bacteroidetes | 100% | 100% | 100% | 100% |
| M2 | Bacteroidetes | 99.31% | 98.94% | 99.41% | 98.04% |
| | Firmicutes | 0.69% | 1.06% | 0.59% | 0.69% |
| M3 | Bacteroidetes | 67.08% | 66.04% | 62.84% | 37.68% |
| | Firmicutes | 0.39% | 0.45% | 0.45% | 0.43% |
| | Proteobacteria | 32.53% | 33.51% | 36.61% | 61.72% |
| M4 | Bacteroidetes | 57.93% | 57.12% | 45.77% | 40.46% |
| | Firmicutes | 0.31% | 0.28% | 0.22% | 0.35% |
| | Proteobacteria | 25.47% | 26.42% | 46.53% | 57.95% |
| | Verrucomicrobia | 16.29% | 16.18% | 7.48% | 1.14% |

TABLE 2

| Strain | Source | Broth | Oxygen sensitivity |
|---|---|---|---|
| Lactobacillus | ATCC 19257 | MRS broth | Facultative |
| cremoris | | | Anaerobe |
| Bacteroides | ATCC 43858 | Chopped | Anaerobic |
| fragilis | | meat broth | |
| Escherichia coli | LF82 | Trypticase | Facultative |
| LF82 | | soy broth | Anaerobe |
| Akkermansia | ATCC | Brain Heart | Anaerobic |
| muciniphila | BAA-835 | Infusion broth | |

Separately, fully assembled devices and free swelling hydrogels were submerged in the containers for 2 hours while being incubated at 37° C. at 100 rpm. Next, the full devices were disassembled. The disassembled components and the free swelling hydrogels were transferred into separate vials with 4 mL of DI water for 2 hours of extraction on an agitator at 100 rpm.

After 2 hours of extraction, DNA extractions were performed on the extraction buffer (DI water) using Invitrogen™ PureLink™ Genomic DNA Mini Kit (Invitrogen, USA). Each bacteria mixture was aliquoted before (“Pre”) and after (“Post”) sampling by the device or the hydrogel. Table 1 shows that all bacterial compositions sampled by full devices resembled the corresponding mixture (both Pre and Post). While the results for the hydrogel alone were less representative, this in-vitro bacterial sampling experiment confirmed the capability of a capsule device to draw a representative sample of gut microbiota.

### In-Vivo Study

An in-vivo evaluation of device function was performed using pigs. The experiment was approved by the Purdue University Animal Care and Use Committee, pursuant to Protocol No. 1911001975A002. Prior to the experiment, the pigs were weighed (each weighing about 40-50 kg) and housed individually in metabolic crates. The pigs were allowed a 5-day acclimation period in their new environment and had ad libitum access to water. Their diet, based on corn and soybean meal, was formulated to meet or exceed the NRC (2012) requirements. The pigs were fed at a daily level of 3 times the estimated maintenance requirement for energy (i.e., 197 kcal of ME/kg of BW0.60; approximately 4% of pig body weight per day), and the daily allotment of feed was provided every morning.

The pigs were fasted for 12 hours prior to administration of the capsules. Two hours after administration of the capsules, the pigs were fed a diet containing a non-digestible ferric oxide marker in order to determine the digestive passage rate. The pigs were then observed at 30-minute intervals to determine the length of time required for the marker to be excreted in the feces (red color).

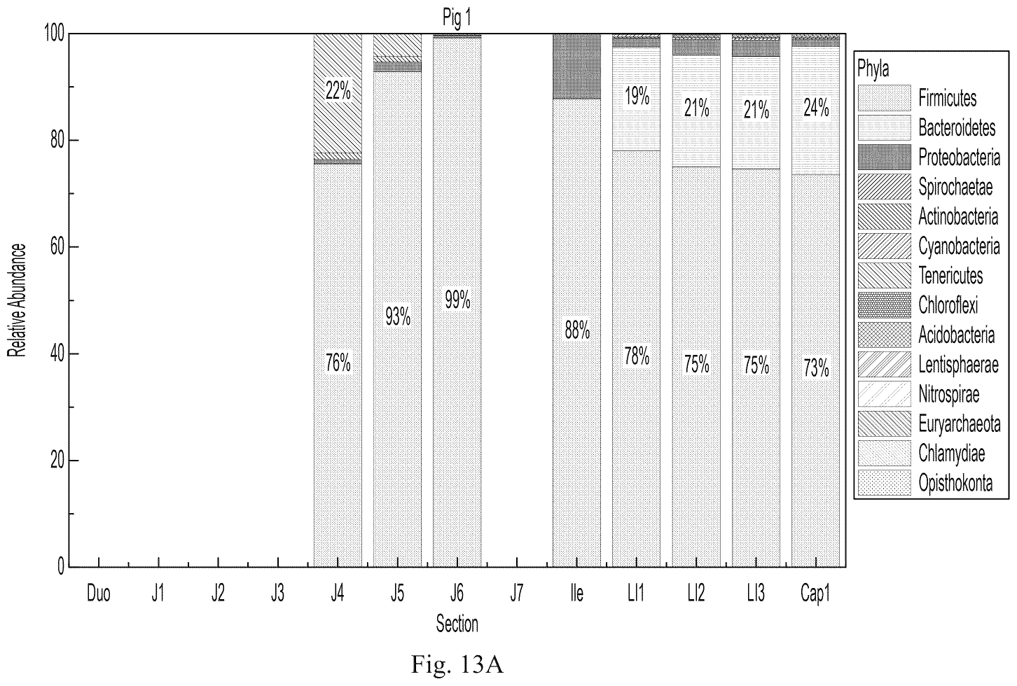

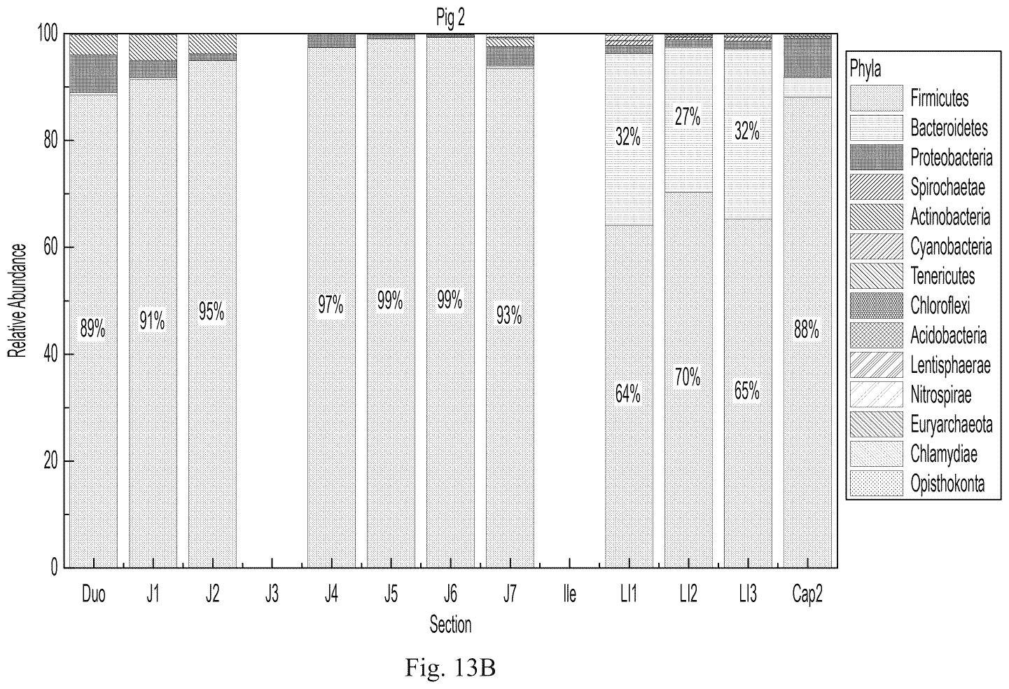

The capsules were administered orally using a balling gun designed for use in pigs. Once the capsules were expelled and collected, each of the pigs was euthanized to measure the pH of various sections within the GI tract. To avoid fluid homogenization and potential errors in pH values, zip-ties were used to isolate the stomach, the small intestine, and the large intestine. To achieve more precise profiling, the small intestine was sectioned in 2-meter increments, (Duo: duodenum, J: jejunum, Ile: ileum as used in FIGS. 13A, 13B) while the large intestine was sectioned in 1-meter increments (LI: large intestine as used in FIGS. 13A, 13B). The fluid content of each section was emptied into a disposable plastic weighing boat, and the pH values were obtained from these fluid contents.

The administered capsules were retrieved from the animal after excretion. After wiping and rinsing the external fecal matter, the capsules were disassembled. The disassembled components were transferred into separate vials with 4 mL of sterile DI water for 2 hours of extraction on an agitator at 100 rpm.

For each of the luminal content samples, the DNA was extracted using the QIAamp PowerFecal Pro DNA Kit (Qiagen, Germany), according to the manufacturer's recommendations. The DNA concentration and quality of each sample were measured with a NanoPhotometer NP60 (Implen, Germany).

The V4 region of the 16S rRNA gene was amplified with PCR primer: 515 Forward, 5′ GTGCCAGCMGCCGCGGTAA, and 806 Reverse, 5′ GGACTACHVGGGTWTCTAAT for all DNA extraction samples. The PCR reactions were performed individually in a total volume of 50 μL using Thermo Scientific™ Phusion High-Fidelity PCR Master Mix (Thermo Scientific, USA) following the manufacturer's recommendations. Verification of the expected amplicon size (˜254 bp) was performed through 2% agarose gel electrophoresis. The PCR products were then purified using Invitrogen™ PureLink™ PCR Purification Kit (Invitrogen Life Technologies, USA).

After checking for sample quality using a NanoPhotometer NP60, PCR products were sent to Purdue Genomics Core Facility for WideSeq (next generation sequencing) analysis. Nextera DNA library preparation kit (Illumina, CA, USA) was used to create sequencing libraries and the multiplexed libraries were sequenced using MiSeq (Illumina, CA, USA) to generate the paired end reads (2×250 bp). Paired end reads from each sample were processed to remove adapters and poor-quality bases. Only the filtered paired reads were used for further processing. Next, reads were re-constructed de novo into a full sequence and were taxonomically assigned using the Silva rRNA database.

The V4 region of 16S rRNA was amplified and sequenced from 17 different luminal content samples from Pig 1 and Pig 2. A mean of 52,413 total reads per sample was obtained by a MiSeq instrument (Illumina, CA, USA). The sequences were processed to remove adapters and poor-quality bases. The filtered reads were re-constructed de novo into a full sequence and were taxonomically assigned using the Silva rRNA database aggregating into 12 phyla. The result was used to create a microbial taxonomic composition profile for Pig 1 and Pig 2.

The results, shown in FIGS. 13A and 13B, showed that the microbiota composition may vary significantly along the porcine intestine, particularly between the small and large intestines. The most dominant phylum throughout the intestine was Firmicutes (64%-99%). The Bacteroidetes phylum increased in the large intestine sections, representing 19%-32% of the total bacteria. The difference between the proportion of Bacteroidetes/Firmicutes in the small intestine and the proportion of Bacteroidetes/Firmicutes in the large intestine could be used as a biomarker to indicate the sampling location of the device.

The two devices administered to Pig 1 and Pig 2 were also collected and analyzed via WideSeq through the same process. Capsule 1 from Pig 1 was found in the feces of Pig 1. Capsule 2 from Pig 2 was not excreted by the time of euthanasia and was found in the large intestine sample while dissecting the GI tract. Capsule 1 had a Firmicutes and Bacteroidetes composition of 73% and 24%, respectively. This result is very similar to the large intestine profile of Pig 1 (75-78% and 19-21%, respectively). Capsule 2 had a lower Bacteroidetes composition (4%). However, the Bacteroidetes composition was still significantly higher than the Bacteroidetes composition in the small intestine sections of Pig 2 (0.02-0.06%). This indicates that the sampling device did draw the sample from the large intestine as intended and as expected.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

What is claimed is:

1. A device for passive sampling of a gastrointestinal tract, the device comprising:
a capsule housing bounding a cavity;
a sampling aperture formed in the capsule housing and providing fluid communication between the cavity and an exterior of the capsule housing;
a sampling hydrogel positioned inside the cavity, wherein upon exposure to a sample fluid, the sampling hydrogel is configured to absorb the sample fluid, expand within the cavity, and store the sample fluid for subsequent analysis;
a sealing member positioned within the cavity between the sampling hydrogel and the sampling aperture, wherein expansion of the sampling hydrogel within the cavity presses the sealing member into engagement with the sampling aperture to seal the cavity; and
a biodegradable coating covering the sampling aperture, the biodegradable coating comprising a plurality of biodegradable coating layers, wherein degradation of the plurality of biodegradable coating layers exposes the sampling aperture to permit fluid flow into the cavity.

2. (canceled)

3. (canceled)

4. (canceled)

5. The device of claim 1, wherein the plurality of biodegradable coating layers comprises a first biodegradable coating layer and a second biodegradable coating layer.

6. The device of claim 5, wherein the first biodegradable coating layer comprises a first biodegradable coating layer configured to degrade at a first predetermined location in the gastrointestinal tract, and the second biodegradable coating layer comprises a second biodegradable coating layer configured to degrade at a second predetermined location in the gastrointestinal tract that is different from the first predetermined location.

7. The device of claim 6, wherein the first biodegradable coating layer is configured to degrade within a small intestine of the gastrointestinal tract, and the second biodegradable coating layer is configured to degrade within a large intestine of the gastrointestinal tract.

8. The device of claim 5, wherein the first biodegradable coating layer is configured to degrade when exposed to a first pH level, and the second biodegradable coating layer is configured to degrade when exposed to a second pH level that is different from the first pH level.

9. The device of claim 8, wherein the first biodegradable coating layer is configured to degrade at a pH greater than 5.5, and the second biodegradable coating layer is configured to degrade at a pH less than 5.5.

10. (canceled)

11. (canceled)

12. (canceled)

13. The device of claim 1, wherein the sampling hydrogel is synthesized from acrylic acid and acrylamide monomers.

14. The device of claim 13, wherein a ratio of acrylic acid monomers to acrylamide monomers is about 10% acrylic acid monomers and about 90% acrylamide monomers.

15. (canceled)

16. (canceled)

17. The device of claim 1, wherein the capsule housing comprises a first housing portion and a second housing portion that are removably attachable to one another to form the capsule housing.

18. The device of claim 17, wherein the first and second housing portions are attachable to one another via a screw interface.

19. The device of claim 17, wherein the sampling aperture is formed in the first housing portion.

20. The device of claim 1, wherein the sampling hydrogel is constructed and arranged to maintain live bacteria present in the sample fluid viable for at least one hour after the cavity is sealed.

21. The device of claim 20, wherein the sampling hydrogel is constructed and arranged to maintain live bacteria present in the sample fluid viable for up to 24 hours after the cavity is sealed.

22. (canceled)

23. (canceled)

24. (canceled)

25. (canceled)

26. A method of manufacturing a device for passive sampling of a gastrointestinal tract, the method comprising:
providing a capsule housing having a cavity and a sampling aperture, the sampling aperture providing fluid communication between the cavity and an exterior of the capsule housing; and
closing the sampling aperture with a biodegradable coating comprising a plurality of biodegradable coating layers, wherein degradation of the plurality of biodegradable coating layers exposes the sampling aperture to permit fluid flow into the cavity.

27. The method of claim 26, wherein closing the sampling aperture with a plurality of biodegradable coating comprises using a drop casting technique to deposit the plurality of biodegradable coating layers within the sampling aperture.

28. The method of claim 27, wherein using a drop casting technique comprises placing a first portion of the capsule housing on a sealing plate, the first portion of the capsule housing including the sampling aperture, and the sealing plate being configured to provide a liquid-tight seal with the sampling aperture, depositing the plurality of biodegradable coating layers within the sampling aperture using a drop casting technique, and removing the first portion of the capsule housing from the sealing plate.

29. The method of claim 26, further comprising drying the plurality of biodegradable coating layers using near infrared radiation.

30. The method of claim 26, wherein the biodegradable coating is disposed over only a portion of the capsule housing.

31. The method of claim 26, wherein the biodegradable coating is disposed within the sampling aperture.

32. The method of claim 31, wherein the plurality of biodegradable coating layers of the biodegradable coating is constrained within the sampling aperture.

33. The method of claim 26, wherein the biodegradable coating is formed as a plane.

34. The method of claim 26, further comprising providing a sampling hydrogel inside the cavity, wherein upon exposure to a sample fluid, the sampling hydrogel is configured to absorb the sample fluid, expand within the cavity, and store the sample fluid for subsequent analysis.

35. The method of claim 26, wherein closing the sampling aperture with a biodegradable coating comprising a plurality of biodegradable coating layers comprises closing the sampling aperture with a first biodegradable coating layer, drying the first biodegradable coating layer using near-infrared radiation, depositing a second biodegradable coating layer onto the first biodegradable coating layer, and drying the second biodegradable coating layer using near-infrared radiation.
